(19) United States
(12) Patent Application Publication
(10) Pub. No.: US 2016/0083309 A1
(43) Pub. Date: Mar. 24, 2016

(54) SYSTEM, METHOD, AND COMPOSITION FOR ENHANCING SOLUTIONS FROM BIOREACTORS FOR PROCESSES INCLUDING LIQUID FERTILIZER PREPARATION AND NUTRIENT EXTRACTION

(71) Applicant: Tinia Pina, New York, NY (US)

(72) Inventor: Tinia Pina, New York, NY (US)

(21) Appl. No.: 14/858,557

(22) Filed: Sep. 18, 2015

Related U.S. Application Data
(60) Provisional application No. 62/052,675, filed on Sep. 19, 2014.

(51) Int. Cl. C05B 17/00; A61L 2/04; C05G 3/00

(52) U.S. Cl. CPC C05B 17/00; C05G 3/0076; A61L 2/04

(57) ABSTRACT

Liquid fertilizers for use in both hydroponic and soil-based growing environments are produced from the food waste processing provided by a bioreactor. The system has a mixture of organic inputs, which are processed using a bioreactor capable of turning the inputs into an aqueous solution. This aqueous solution is then formulated into a composition that can be bottled and distributed as a product within the consumer product and agriculture industry.

### CROSS-REFERENCE TO RELATED APPLICATIONS

This application claims priority to U.S. Provisional Application No. 62/052,675, filed Sep. 19, 2014, which is herein incorporated by reference in its entirety.

### FIELD OF THE INVENTION

The present invention relates to systems, methods, compositions, kits, and containers for organic-based nutrient products prepared from a food supply chain waste, in particular relating to systems, methods, compositions, kits, and containers for agricultural systems such as, for example, hydroponic and soil-based systems.

### BACKGROUND OF THE INVENTION

Waste remains an important issue, not only in developing countries, but world-wide. Indeed, there is an ever-increasing global awareness and appreciation for better waste management and reuse of materials. For example, the increasing demand for food production without overtaxing land, natural resources, and/or destroying wildlife habitat, along with other drivers, calls for continuing research and development aimed at finding creative and cost-effective solutions for the production of added-value materials.

There is a need for systems, methods, compositions, kits, and containers for the production of nutrient materials from waste.

### SUMMARY OF THE INVENTION

In one aspect, the present invention provides a method for preparing a composition comprising nutrients. The method comprises subjecting an effluent from a bio-reactor to conditions of time and temperature to inactivate substantially all microbes that may be present in the effluent; and optionally, contacting the effluent with a source of a nutrient to adjust a level of the nutrient in the effluent.

In another aspect, the present invention provides a method for preparing a composition comprising nutrients. The method comprises determining a level of a nutrient in an effluent from a bio-reactor, contacting the effluent with a source of the nutrient to adjust the level of the nutrient in the effluent; and subjecting the effluent to conditions of time and temperature to inactivate substantially all microbes that may be present in the effluent.

In other aspects, the present invention provides a composition comprising nutrients prepared according to a method comprising subjecting an effluent from a bio-reactor to conditions of time and temperature to inactivate substantially all microbes that may be present in the effluent; and optionally, contacting the effluent with a source of a nutrient to adjust a level of the nutrient in the effluent.

In some aspects, the present invention provides a composition comprising nutrients prepared according to a method comprising determining a level of a nutrient in an effluent from a bio-reactor; contacting the effluent with a source of the nutrient to adjust the level of the nutrient in the effluent; and subjecting the effluent to conditions of time and temperature to inactivate substantially all microbes that may be present in the effluent.

In one aspect, a composition comprising a food waste extract and a plant-based ingredient, wherein the composition is suitable for use in a system for growing a plant, is provided by the present invention.

In another aspect, the present invention provides kits or containers comprising the compositions of the present invention.

In other aspects, the present invention provides a system comprising a first container for receiving an effluent from a bio-reactor that composts a bio-compostable material.

### BRIEF DESCRIPTION OF THE DRAWINGS

FIG. 1 shows a schematic view of a process flow diagram according to one embodiment of the present invention.

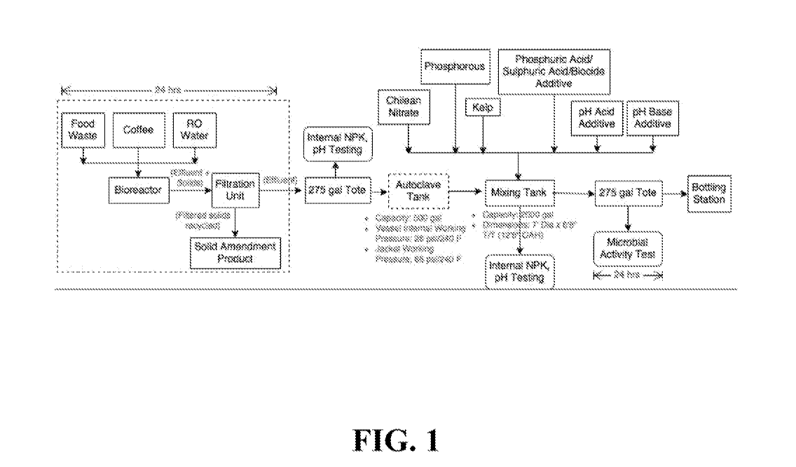

FIG. 2 shows a flowchart of process outline according to another embodiment of the present invention.

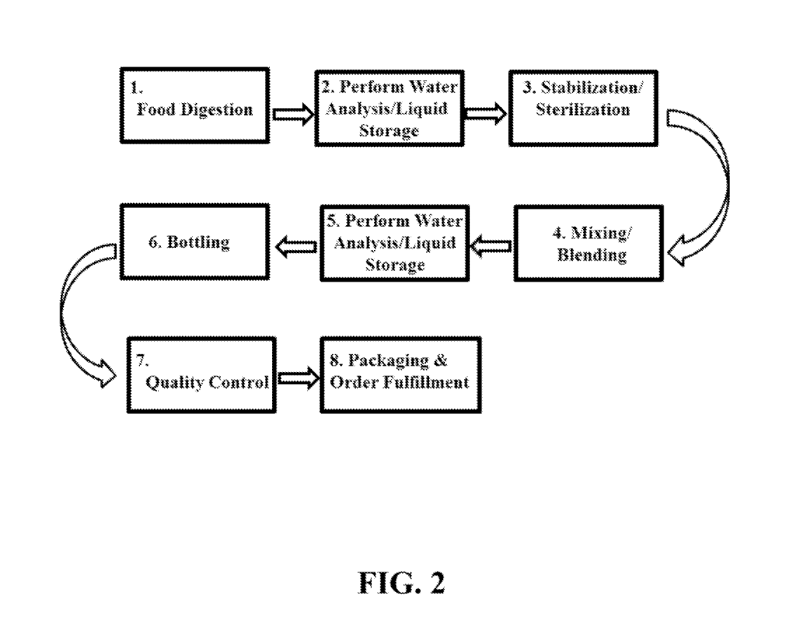

### DETAILED DESCRIPTION OF THE INVENTION

The present invention is applicable but not limited to the use of organic waste for preparing compositions comprising nutrients, in particular to the use of food waste for preparing an aqueous food composition for a plant.

The term “plant,” as used herein, refers to an organism belonging to the kingdom Plantae (i.e., any genus species in the Plant Kingdom). This includes but is not limited to fruit-producing plants, vegetable-producing plants, row crops, vegetable crops, flowers, trees, herbs, bushes, grasses, vines, ferns, mosses and green algae. Thus, unless the context specifically states or requires otherwise, the term refers to both living and once living such organisms as well as to both monocotyledonous plants, also called monocots, and dicotyledonous plants, also called dicots.

Examples of plants include but are not limited to corn, potatoes, roses, apple trees, sunflowers, wheat, rice, bananas, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, brassica leafy vegetables (e.g., broccoli, broccoli raab, Brussels sprouts, cabbage, Chinese cabbage (Bok Choy and Napa), cauliflower, cavalo, collards, kale, kohlrabi, mustard greens, rape greens, and other brassica leafy vegetable crops), bulb vegetables (e.g., garlic, leek, onion (dry bulb, green, and Welch), shallot, and other bulb vegetable crops), citrus fruits (e.g., grapefruit, lemon, lime, orange, tangerine, citrus hybrids, pummelo, and other citrus fruit crops), cucurbit vegetables (e.g., cucumber, citron melon, edible gourds, gherkin, muskmelons (including hybrids and/or cultivars of cucumis melons), water-melon, cantaloupe, and other cucurbit vegetable crops), fruiting vegetables (including eggplant, ground cherry, pepino, pepper, tomato, tomatillo, and other fruiting vegetable crops), grape, leafy vegetables (e.g., romaine), root/tuber and conn vegetables (e.g., potato), and tree nuts (e.g., almond, pecan, pistachio, and walnut), berries (e.g., tomatoes, barberries, currants, elderberries, gooseberries, honeysuckles, mayapples, nannyberries. Oregon-grapes, see-buckthorns, hackberries, bearberries, lingonberries, strawberries, sea grapes, blackberries, cloudberries, loganberries, raspberries, salmonberries, thimbleberries, and wine-berries), cereal crops (e.g., corn, rice, wheat, barley, sorghum, millets, oats, ryes, triticales, buckwheats, fonio, and quinoa), pome-fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), libber crops (e.g., hemp, cotton), ornamentals, etc.

The plant may in some embodiments be a household/domestic plant, a greenhouse plant, an agricultural plant, a horticultural plant, a silvicutural and/or an ornamental plant.

In another embodiment, the plant is a member (e.g., Cannabis sative) of the genus Cannabis.

The term “plant” is also intended to include any plant propagules.

The term “plant part,” as used herein, refers to any part of a plant including but not limited to the shoot, root, stem, seeds, stipules, leaves, petals, flowers, ovules, bracts, branches, petioles, internodes, bark, wood, tubers, pubescence, tillers, rhizomes, fronds, blades, pollen, stamen, fruit and the like.

In one aspect, the present invention provides a method for preparing a composition comprising nutrients. The method comprises

subjecting an effluent from a bioreactor to conditions of time and temperature to inactivate substantially all microbes that may be present in the effluent; and

optionally, contacting the effluent with a source of a nutrient to adjust a level of the nutrient in the effluent.

The term “bioreactor” (also known as “biochemical reactor”), as used herein, is intended to be broad and refers to any enclosed or partially enclosed chamber(s) and/or system(s) adapted and configured to have a transformation or conversion (e.g., composting) of a substrate take place therein carried out by microbes and/or enzymes. Thus, unless specifically stated otherwise, the term “bioreactor” is not limited to any particular type or configuration and includes but is not limited to a food waste liquefier (also referred to as bio-digesters or wet systems), fluidized bed reactors, plug flow reactors, batch sequencing reactors, completely stirred tank reactors, granular activated sludge reactors, and combinations thereof, and other types of reactor systems that promote transformation or conversion of organic and/or inorganic substrates by microbes and/or enzymes.

The term “derived from,” as used herein, is intended to refer to the origin or source. Thus, the term “derived from” indicates that one specified material finds its origin or source in another specified material or has features that can be described with reference to the another specified material. For example, a transformation or conversion of a substrate in a bioreactor may give rise to a particular nutrient, and thus, the particular nutrient is said to have been “derived from” the substrate.

In one embodiment, the bioreactor comprises an aerobic condition sufficient to compost a substrate therein to form an effluent comprising at least one nutrient derived from the substrate.

In another embodiment, the bioreactor comprises an anaerobic condition sufficient to compost a substrate therein to form an effluent comprising one or more nutrients derived from the substrate.

In other embodiments, the bioreactor comprises a hybrid system comprising an aerobic condition before or after an anaerobic condition, wherein the bioreactor composts a substrate therein to form an effluent comprising at least one nutrient derived from the substrate.

In some embodiments, the bioreactor is a food waste liquefier.

In one embodiment, the bioreactor is Eco-Safe Digestor™ (VetUS Environmental Services, Augustine, Fla.).

Examples of bioreactors include but are not limited to those described in U.S. Pat. Nos. 7,735,761, 7,762,713, 8,789,779, 8,927,266 and in US Patent Publication No. 20130118969, each of which is herein incorporated by reference for its disclosure of a bioreactor.

The term “effluent,” as used herein, refers to an aqueous composition that is present in a bioreactor, or that is derived directly or indirectly from the bioreactor, following a transformation or conversion of a substrate having taken place therein the bioreactor for a period of time. Thus, unless specifically stated otherwise, the term “effluent” includes further processed downstream forms of an initial effluent. For example, unless specifically stated otherwise, a downstream filtrate is intended to be encompassed by the term “effluent” where the filtrate is produced by filtration of a liquid composition that at some point originated directly from inside the bioreactor following a transformation or conversion of a substrate having taken place therein the bioreactor for a period of time. In other words, unless specifically stated otherwise, the term effluent” is not intended to be limited to only the initial effluent that comes out of the bioreactor.

Generally, the bioreactor composts a substrate to form an effluent comprising at least one nutrient derived from the substrate. For example, the substrate (e.g., food waste) for the bioreactor, optionally along with other bio-compostable material, is introduced to the bio-reactor. Facilitated by a sufficient concentration of natural- and/or biologically produced microorganisms, enzymes, and/or other material (such as, for example, humic acid, coffee grinds, and/or wood chips) that may be present with the substrate and/or that may be in or separately introduced into the bioreactor, the substrate undergoes a transformation or conversion (e.g., composts) in the bioreactor for a period of time under conditions sufficient to form the effluent.

Examples of microorganism that may be utilized in the bioreactor include but are not limited to microorganisms of the genera of Saccharrmyce, Aspergillus, Lactobacillus, Bacillus, Klebsiella, Enterobacter, Bacteroides, Fusobacterium, Desulfbvibrio, Desulfuromonas, Clostridium, Desulfototmaculum, Sporosarcina, Pseudomonas, Veillonella, Acidaminococcus, Methwnobacterium, Methanococcus, Archaeoglobus, and combinations thereof.

In one embodiment, the bioreactor comprises at least one microorganism selected from the group consisting of Saccharomyces cerevisiae, Lactobacillus paracasei, Bacillus subtilis, and Aspergillus oryae.

In another embodiment, the bioreactor comprises Saccharomyces cerevisiae, Lactobacillus paracasei, Bacillus subtills, and Aspergillus oryzae.

In some embodiments, the bioreactor comprises wood chips and a blend of Saccharomvces cerevisiae. Lactobacillus paracasei, Bacillus subtilis, and Aspergillus orzae.

In some embodiments, an aqueous solution (e.g. water, buffer, etc.) is introduced inside a chamber of the bio-reactor to create an environment (e.g., aerobic, anaerobic) for the biodegradation of the food waste to occur.

In some embodiments, the aqueous solution is treated before entering the bioreactor.

For example, reverse osmosis (RO) and nanofiltration (NF) are filtration methods that, in some embodiments, may be used to treat a solution (e.g., water (e.g., grey, potable, industrial, tap)) by removing or reducing total dissolved solids (TDS) and/or residual organic compounds from various water sources such as, for example, from natural water sources, municipal water supply and/or commercial and/or industrial water and/or effluents.

RO relies on a diffusive mechanism to separate relatively large molecules and ions from a solution by applying pressure to the solution on one side of a semipermeable membrane. NF is typically a cross-flow filtration technology which ranges somewhere between ultrafiltration (UF) and reverse osmosis. The filtration process takes place on a selective separation layer formed by a semipermeable membrane. Both RO and NF may be a pressure driven separation process. The driving force of the separation process is the pressure difference between the feed (retentate) and the filtrate (permeate) side at the separation layer of the membrane.

RO membrane modules can be supplied in a variety of properties. So-called seawater membranes are used to desalinate seawater (equivalent to approximately 35,000 ppm NaCl) at pressure of about 800 psi to about 1500 psi. This type of membrane will retain over 99% of incident salt. So-called brackish water membranes operate at lower pressures in waters of lower ionic strength. They will have relatively lower inherent retention of salt ions, but have a higher permeability and when properly engineered, will operate economically. NF membranes are so-called “loose” reverse osmosis membranes which retain multivalent ions and species of about 400 molecular weight (MW) or greater. NF generally pass a high percentage of monovalent ions. They have relatively higher permeability than the previously described membranes.

In some embodiments, in a RO process, a continuous flow of feed water contacts across one side of the RO membrane at an elevated pressure. The pressure is above the osmotic pressure of the feed water, generally multiples of the osmotic pressure. Purified water passes through the membrane to the low pressure side of the process as permeate. The retained salts and organic matter removed from the feed water are concentrated in the remaining water, that is, the water that does not exit as permeate. This is the reject stream, which flows to be processed or disposed of, depending on the desired configuration of the RO process.

In other embodiments, once the food waste is sufficiently composted and small enough to pass through a part of the bioreactor, e.g. through a filter or screen (e.g., a filter or screen located in the base of the bio-reactor), the aqueous material can be removed or washed out as an effluent comprising at least one nutrient derived from the food waste.

In some embodiments, the substrate comprises a bio-compostable material.

The term “bio-compostable material,” as used herein, includes any variety of organic substrates, which may be animal and/or non-animal (e.g., fruits, vegetables) in origin, and which through microbial action and/or decay and/or oxidation can be transformed or converted to provide one or more suitable nutrients for various purposes including but not limited to for agricultural purposes, or to some other form for disposal or use.

In one embodiment, the substrate comprises a food waste, wherein the effluent comprises at least one nutrient derived from the food waste.

The invention relates to all food waste including but not limited to plant and/or plant part solid and/or liquid wastes, animal solid and/or liquid wastes, and/or combined waste (e.g., a mixture of any of the foregoing with each other or other nonfood waste).

Food waste for purposes of this invention includes but is not limited to uneaten food, food preparation wastes at or from: residences and/or commercial establishments (e.g., a food establishment such as, for example, restaurants, fast food chains, etc.), institutional sources such as school cafeterias, nursing homes, correctional facilities, etc., industrial sources such as factory lunchrooms, etc., industrial food preparation and packaging, commissaries (prepares food for sale through its own food outlets), food processing facilities (manufacturers, packages, labels or stores food but does not sell directly to consumers), and combined waste (e.g. a mixture of any of the foregoing with each other or other nonfood waste) hence forth collectively referred to as food waste.

Food supply chain waste is the organic material produced for human consumption that may have been discarded, lost or degraded e.g. at the manufacturing and retail stages, including waste arising from degradation by pests or spoilage. Food waste is produced at most if not all stages of the food supply chain, including at the retail and consumer stage. The agro-food supply chain encompasses a broad variety of manufacturing processes that generate accumulative quantities of different agro-food supply chain waste, in particular organic residues.

In some embodiments, the food waste is a product of one or more upstream processing schemes including, but not limited to, sorting, separation, screening, purification, smashing, chopping, mulching, grinding, mixing, heating, cooling, etc.

For example, mulching, screening and/or sorting may be carried out to remove any objects from the food waste that are greater in size than a pre-determined maximum size prior to introduction of the food waste into the bioreactor.

In another embodiment, a draining step may be included comprising draining for a pre-determined amount of time, draining until a pre-determined amount of liquid is collected, or draining until a rate of flow of the liquid is reduced to a predetermined minimum rate.

In some embodiments, a mixing step may be included and may be accomplished using any desired mixing device or process. For example, a mixing may occur within an enclosed container. Alternatively, the mixing may occur in an open area.

Such one or more upstream processing schemes may be performed at or in the vicinity of the site where the bioreactor is located, or they may be carried out at a different location(s) and the final product (i.e., substrate) transported to the site where the bioreactor is located.

Methods and systems for upstream processing of waste such as, for example, waste that includes a mixture of wet organic material and dry organic material and optionally inorganic material are known in the art. One such method and system is disclosed in U.S. Pat. No. 8,398,006 to Gitschel, which is incorporated herein by reference in its entirety. The system and method according to U.S. Pat. No. 8,398,006 mechanically separates the mixed solid waste to produce a wet organic stream enriched in wet organics and a dry organic stream enriched in dry organics, and each stream is separately processed to convert at least a portion of each stream into a product for further downstream handling/processing.

In some embodiments, the bioreactor is at or in a vicinity of a site comprising the food waste. In one such embodiment, the food waste that is generated and/or further processed (e.g., sorted) onsite may be used as the substrate for the bioreactor.

Thus, for example, the bioreactor may be located at or in the vicinity of residences and/or commercial establishments (e.g., a food establishment such as, for example, restaurants, fast food chains, etc.), institutional sources such as school cafeterias, nursing homes, correctional facilities, etc., industrial sources such as factory lunchrooms, etc., industrial food preparation and packaging, commissaries (prepares food for sale through its own food outlets), food processing facilities (manufacturers, packages, labels or stores food but does not sell directly to consumers), landfills, and the like.

In other embodiments, the bioreactor is located at a remote location relative to the location of the food waste.

In one embodiment, the food waste comprises a food supply chain waste.

In another embodiment, the food supply chain waste comprises an agro-food supply chain waste.

In other embodiments, the food waste comprises a plant or plant part.

In another embodiment, the food waste comprises a fruit.

In one embodiment, the food waste comprises a vegetable.

In some embodiments, the substrate for the bioreactor comprises food waste, wherein the food waste comprises a total plant or plant part content of at least 1% (w/w), illustratively, about 1% to about 100%, about 1.0% to about 95%, about 20% to about 90%, about 30% to about 80%, about 40% to about 70%, about 50% to about 60% (w/w).

In one embodiment, the substrate for the bioreactor comprises food waste, wherein the food waste comprises a total plant or plant part content of 50% or more (w/w).

In another embodiment, the substrate for the bioreactor comprises food waste, wherein the food waste comprises a total plant or plant part content of 95% or more (w/w).

In one embodiment, the substrate for the bioreactor comprises food waste, wherein the food waste comprises a total plant or plant part content of 99% (w/w).

In another embodiment, the substrate for the bioreactor comprises food waste, wherein the food waste comprises a total plant or plant part content of 100% (w/w).

Any combination of plant or plant part may be included in the food waste to be used as the substrate for the bioreactor, or the substrate may include only one type of plant or plant part.

The types and/or amounts of plant or plant part selected to be used as the substrate for the bioreactor may influence, among other parameters, a nutrient content of the effluent that forms following the transformation or conversion of the substrate.

In some embodiments, the substrate for the bioreactor comprises meat, poultry, fish, fruit, vegetables, plants, grains, dairy, eggs, or eggshells, and combinations thereof.

In other embodiments, the substrate for the bioreactor does not contain, or is sorted so as not to contain, such items as bones, seeds (e.g., over ½ inch diameter), corn husks and cobs, flour, dough, cooking oils, grease and fats.

In one embodiment, the substrate does not contain animal waste (e.g., manure) and/or other animal-based product such as meat, skin, bone and the like.

In another embodiment, the substrate does not contain packaging (e.g., cardboard, metal, plastic, etc.).

In some embodiments, the food waste comprises one or more plants or plant parts, wherein the one or more plants or plant parts are a vegetable.

In another embodiment, the food waste comprises one or more plants or plant parts, wherein the one or more plants or plant parts are a fruit.

In some embodiments, the food waste comprises one or more plants or plant parts, wherein the one or more plants or plant parts are a vegetable.

In one embodiment, the food waste comprises one or more plants or plant parts, wherein the one or more plants or plant parts comprise fruits and vegetables.

In some embodiments, the at least one nutrient derived from the food waste is suitable for use as a nutrition for a plant.

In another embodiment, the at least one nutrient derived from the food waste is nitrogen (N), phosphorus (P), or potassium (K).

In other embodiments, the effluent comprises at least one nutrient derived from the food waste, wherein the total at least one nutrient derived from the food waste content of the effluent is at least about 0.01% by weight, illustratively, at least about 0.01%, at least about 0.1%, at least about 0.5%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.1%, at least about 3.2%, at least about 3.3%, at least about 3.4%, at least about 3.5%, at least about 3.6%, at least about 3.7%, at least about 3.8%, at least about 3.9%, at least about 4%, about 4.1%, at least about 4.2%, at least about 4.3%, at least about 4.4%, at least about 4.5%, at least about 4.6%, at least about 4.7%, at least about 4.8%, at least about 4.9%, at least about 5%, about 5.1%, at least about 5.2%, at least about 5.3%, at least about 5.4%, at least about 5.5%, at least about 5.6%, at least about 5.7%, at least about 5.8° %, at least about 5.9%, at least about 6%, about 6.1%, at least about 6.2%, at least about 6.3%, at least about 6.4%, at least about 6.5%, at least about 6.6%, at least about 6.7%, at least about 6.8%, at least about 6.9%, at least about 7%, about 7.1%, at least about 7.2%, at least about 7.3%, at least about 7.4%, at least about 7.5%, at least about 7.6%, at least about 7.7%, at least about 7.8%, at least about 7.9%, and at least about 8% by weight.

In other embodiments, the effluent comprises nitrogen derived from the food waste, wherein the total nitrogen derived from the food waste content of the effluent is at least about 5% by weight.

In some embodiments, the effluent comprises phosphorus derived from the food waste, wherein the total phosphorus derived from the food waste content of the effluent is at least about 5% by weight.

In one embodiment, the effluent comprises potassium derived from the food waste, wherein the total potassium derived from the food waste content of the effluent is at least about 5% by weight.

In another embodiment, the effluent comprises a total combined nitrogen, phosphorus, and potassium (N—P—K) content of at least 5% by weight that is derived from the food waste.

In other embodiments, the effluent comprises nitrogen derived from the food waste, wherein the total nitrogen derived from the food waste content of the effluent is at least about 6% by weight.

In some embodiments, the effluent comprises phosphorus derived from the food waste, wherein the total phosphorus derived from the food waste content of the effluent is at least about 6% by weight.

In one embodiment, the effluent comprises potassium derived from the food waste, wherein the total potassium derived from the food waste content of the effluent is at least about 6% by weight.

In another embodiment, the effluent comprises a total combined nitrogen, phosphorus, and potassium (N—P—K) content of at least 6% by weight that is derived from the food waste.

In some embodiments, the effluent comprises a total nitrogen content of about 1 mg/L to about 100,000 mgL, illustratively, about 1 mg/L to about 100,000 mg/L, about 10 mg/L to about 90,000 mg/L, about 50 mg/L to about 90,000 mg/L, about 100 mg/L to about 80,000 mg/L, about 500 mg/L to about 70,000 mg/L, about 1000 mg/L to about 60,000 mg/L, about 5000 mg/L to about 50,000 mg/L, about 10,000 mg/L to about 40,000 mg/L, and about 20,000 mg/L to about 30,000 mg/L.

In other embodiments, the effluent comprises a total nitrogen content of at least about 1 mg/L, illustratively, at least about 1 mg/L, at least about 10 mg/L, at least about 100 mg/L, at least about 500 mg/L, at least about 1000 mg/L, at least about 5000 mg/L, at least about 10,000 mg/L, at least about 20,000 mg/L, at least about 30,000 mg/L, at least about 40,000 mg/L, at least about 50,000 mg/L, at least about 60,000 mg/L, at least about 70,000 mg/L, at least about 80,000 mg/L, at least about 90,000 mg/L, and at least about 100,000 mg/L.

In one embodiment, the effluent comprises a total phosphorus content of about 0.1 mg/L to about 10,000 mg/L, illustratively, about 0.1 mg/L to about 10,000 mg/L, about 1 mg/L to about 9,000 mg/L, about 50 mg/i to about 8,000 mg/L, about 100 mg/L to about 7,000 mg/L, about 500 mg/L to about 5,000 mg/L, about 1000 mg/L to about 4,000 mg/L, and about 2000 mg/L to about 3,000 mg/L.

In another embodiment, the effluent comprises a total phosphorus content of at least about 0.1 mg/L, illustratively, at least about 0.1 mg/L, at least about 1 mg/L, at least about 10 mg/L, at least about 100 mg/L, at least about 500 mg/L, at least about 1000 mgL, at least about 2000 mg/L, at least about 3,000 mg/L, at least about 4,000 mg/L, at least about 5,000 mg/L, at least about 6,000 mg/L, at least about 7,000 mg/L, at least about 8,000 mg/L, at least about 9,000 mg/L, at least about 10,000 mg/L, at least about 20,000 mg/L, and at least about 30,000 mg/L.

In some embodiments, the effluent comprises a total potassium content of about 1 mg/L to about 100,000 mg/L, illustratively, about 1 mg/L to about 100,000 mg/L, about 10 mg/L to about 90,000 mg/L, about 50 mg/i to about 90,000 mg/L, about 100 mg/L to about 80,000 mg/L, about 500 mg/L, to about 70,000 mg/L, about 1000 mg/L, to about 60,000 mg/L, about 5000 mg/L to about 50,000 mg/L, about 10,000 mg/L to about 40,000 mg/L, and about 20,000 mg/L to about 30,000 mg/L.

In other embodiments, the effluent comprises a total potassium content of at least about 1 mg/L, illustratively, at least about 1 mg/L, at least about 10 mg/L, at least about 100 mg/L, at least about 500 mg/L, at least about 1000 mg/L, at least about 5000 mg/L, at least about 10,000 mg/L, at least about 20,000 mgL, at least about 30,000 mg/L, at least about 40,000 mg/L, at least about 50,000 mg/L, at least about 60,000 mg/L, at least about 70,000 mg/L, at least about 80,000 mg/L, at least about 90,000 mg/L, and at least about 100,000 mg/L.

In one embodiment, the effluent from the bioreactor is subjected to conditions of time and temperature to inactivate substantially all microbes that may be present in the effluent.

The terms “microbe(s)” or “microorganism(s)” are used herein interchangeably to refer to organisms having a single cell, cell clusters or no cell at all, and include, for example, bacteria, fungi, archaea, protists, and the like, including spores thereof. Thus, unless specifically stated otherwise, these terms are intended to encompass microbes that are present with the food waste as well as microbes that may be introduced into the bioreactor to facilitate the decomposition of the food waste inside the bioreactor.

The terms “inactivate(s),” “inactivation.” or “inactivating” as used herein, are intended to be broad and refer to the physical removal of the microbes present in a composition and/or to altering the microbes present in a composition such to put the microbes in a state in which the microbes are non-culturable. Thus, unless specifically stated otherwise, the term “inactivate” in the context of, for example, subjecting an effluent from a bio-reactor to conditions of time and temperature to “inactivate” substantially all microbes that may be present in the effluent includes but is not limited to e.g., a step of filtering of the effluent in order to physically remove substantially all microbes that may be present in the effluent.

The phrase “substantially all” as used herein refers to at least about 70%, illustratively, at least about 70%, at least about 80%, at least about 90% at least about 91%, at least about 92%, at least about 93%, at least about 94%, at least about 95%, at least about 96%, at least about 97%, at least about 98%, at least about 99%, at least about 99.99%, at least about 99.9999%, and 100%.

The conditions of time and temperature that are needed to inactivate substantially all microbes that may be present in the effluent may vary depending on a number of factors including the method/technique used to inactive.

Exemplary methods contemplated to be applicable to the present invention for inactivating microbes include but are not limited to biocide/preservative treatment, heating (e.g., autoclaving), filtration (e.g., 0.22 μm and/or 0.45 μm membrane filtration), cold pasteurization (e.g., high pressure processing (HPP)), irradiation (e.g., ultraviolet (UV), gamma), solvent/detergent treatment, disinfection, etc. Any one of such methods/techniques may be employed exclusively or in various combinations with others as appropriate.

In some embodiments, the step of subjecting an effluent from a bioreactor to conditions of time and temperature to inactivate substantially all microbes that may be present in the effluent comprises killing at least about 70%, at least about 80%, at least about 90%, at least about 95%, at least about 99%, at least about 99.99%, at least about 99.9999%, or 100% of the microbes present in the effluent.

In other embodiments, the methods of the present invention provide at least a 1 log, at least a 2 log, at least a 3 log, at least a 4 log, or at least a 5 log or more reduction to complete inactivation of microbes in the effluent.

In one embodiment, the step of subjecting an effluent from a bioreactor to conditions of time and temperature to inactivate substantially all microbes that may be present in the effluent comprises heating the effluent at a temperature and for a period of time sufficient to inactivate substantially all microbes present in the effluent.

Heating a composition at a temperature and for a period of time sufficient to inactivate substantially all microbes present in the composition is a standard technique that is well known to one of ordinary skill in the art. The parameters of temperature and time may be varied in accordance with standard procedures known in the art.

For example, heating may be carried out by raising the temperature of the effluent to at least 115° C. (Celsius) (i.e., about 240° F. (Fahrenheit)) for at least 10 minutes, illustratively, to at least 115° C., 120° C., 125° C., or 130° C., or more for at least 10, 11, 12, 13, 14, 15, 16, 17, 18, 19, 20, 25, 30, 40, 50, or 60 minutes or more as necessary to inactivate substantially all microbes present in the effluent.

For example, in some embodiments, the heating process can be carried out in autoclaves using steam or overheated water which comes in contact with the outer surfaces of the containers of the effluent and heat the effluent to the desired temperature and for the time sufficient to inactivate substantially all microbes present in the effluent. Such systems as known in the art may be configured with a steam or overheated water generation system as a heat source, and may include pressure regulation systems for balancing internal pressures in the containers and involve the use of hermetic receptacles with adequate wall thicknesses to withstand the mechanical stresses caused by such pressures.

In another embodiment, the step of subjecting comprises heating the effluent at a temperature of about 240° F. for a period of time sufficient to inactivate substantially all microbes present in the effluent.

In one embodiment, the period of time is about 15 minutes to about 1 hour.

In other embodiments, the step of heating is carried out in a pressure tank having a vessel internal working pressure of about 26 psi (pressures per square inch) and a jacket working pressure of about 66 psi, wherein the effluent is subjected to a temperature of at least 240 OF for a period of time sufficient to inactivate substantially all microbes present in the effluent.

In another embodiment, the effluent is heated to about 240° F. at least about 26 psi and held for at least 15 minutes to effect complete sterilization of bacteria and spores that may be present in the effluent.

A variety of pressure tanks (e.g., autoclave) are available in the art including but not limited to stainless steel jacketed tanks with mixers such as, for example, tanks manufactured by Cherry Burrell.

In one embodiment, the step of heating the effluent at a temperature and for a period of time sufficient to inactivate substantially all microbes present in the effluent is performed outside of the bioreactor.

In another embodiment, the bioreactor is configured to perform the step of heating the effluent at a temperature and for a period of time sufficient to inactivate substantially all microbes present in the effluent.

In some embodiments, the step of subjecting an effluent from a bioreactor to conditions of time and temperature to inactivate substantially all microbes that may be present in the effluent comprises contacting the effluent with a biocide at a temperature and for a period of time sufficient to inactivate substantially all microbes that may be present in the effluent.

In another embodiment, the step of subjecting an effluent from a bioreactor to conditions of time and temperature to inactivate substantially all microbes that may be present in the effluent comprises contacting the effluent with a preservative at a temperature and for a period of time sufficient to inactivate substantially all microbes that may be present in the effluent.

Examples of suitable preservatives include but are not limited to HydroStat™ (Hydros, Inc., Environmental Diagnostics, Bourne, Mass.), β-propiolactone, thimerosal, and propylene glycol, or any other suitable preservative having bacteristat activity.

In one embodiment, the preservative comprises HydroStat™.

In another embodiment, at least about 10 ml of HydroStat™ is added per liter of the effluent, and allowed to inactivate microbes for at least 24 hours before use.

In some embodiments, the step of subjecting an effluent from a bioreactor to conditions of time and temperature to inactivate substantially all microbes that may be present in the effluent comprises contacting the effluent with a composition comprising sulfuric acid and/or phosphoric acid at a temperature and for a period of time sufficient to inactivate substantially all microbes that may be present in the effluent.

One of ordinary skill in the art will recognize that different biocides/preservatives may be added in different effective amounts based on the strength or ability of a biocide(s)/preservative(s) to deactivate or kill microbes.

In other embodiments, the step of subjecting an effluent from a bioreactor to conditions of time and temperature to inactivate substantially all microbes that may be present in the effluent comprises filtering the effluent at a temperature and for a period of time sufficient to physically remove substantially all microbes that may be present in the effluent.

In one embodiment, filtration is carried out using 0.22 μm and/or 0.45 μm membrane filters. Said filtering step is to filter out e.g., bacteria and other microorganisms that may be present in the effluent.

In another embodiment, the step of subjecting an effluent from a bioreactor to conditions of time and temperature to inactivate substantially all microbes that may be present in the effluent comprises exposing the effluent to ultraviolet (UV) light at a temperature and for a period of time sufficient to inactivate substantially all microbes present in the effluent.

For example, the effluent may be subjected to sterilization through UV light (e.g., far-UV range of 254 nm) exposure for about 1 to about 2 hours.

In other embodiments, a method for preparing a composition comprising nutrients further comprises: optionally, contacting an effluent from a bioreactor with a source of a nutrient to adjust a level of the nutrient in the effluent.

In some embodiments, the nutrient includes but not limited to nitrogen, phosphorus, potassium, calcium, magnesium, boron, chlorine, copper, manganese, molybdenum, sulfur, iron, zinc, silicon, etc.

There is also a large variety of other compounds of nutritional value to a plant that stimulate and/or optimize the growth of a plant including but not limited to beneficial bacteria and fungi, enzymes, yeast extracts, carbohydrates, amino acids, hormones, vitamins, organic ingredients, organic extracts, etc.

Numerous sources are available that can serve as the source of a particular nutrient or a combination of nutrients. For example, the source can include but is not limited to coffee grounds, kelp, seaweed, Chilean nitrate, potash, ash, sawdust, wood shavings, humates, dextrose, gypsum, carbonized limestone, liquid fish, ammonium sulfate, monoammonium phosphate, diammonium phosphate, calcium carbonate, calcium chelate, calcium nitrate, magnesium phosphate, magnesium sulfate, monopotassium phosphate, potassium bicarbonate, potassium nitrate, potassium sulfate, potassium silicate, urea, urea-ammonium nitrate solution (UAN) (e.g., UAN 28, 30, and 32% by weight nitrogen), etc., and combinations thereof.

In one embodiment, the nutrient is a plant nutrient.

In another embodiment, the nutrient is selected from the group consisting of: H₂PO₄⁻, SO₄²⁻, NH₄⁺, Ca²⁺, K⁺ and Mg²⁺.

In other embodiments, the nutrient is nitrogen (N).

In some embodiments, the nutrient is phosphorus (P).

In another embodiment, the nutrient is potassium (K).

The nutrient content of an effluent from a bioreactor can be analyzed using a variety of conventional analysis methods and/or devices available to one of ordinary skill in the art. Consequently, if any of the nutrient (e.g., N—P—K) values are below a desired level, they may be adjusted to the desired level. Accordingly, in other embodiments, the method further comprises determining a level of a nutrient in an effluent from a bio-reactor.

The nutrient content of an effluent from a bioreactor can be analyzed using a variety of conventional analysis methods and/or devices available to one of ordinary skill in the art.

For example, the contents of one or more nutrients (e.g., nitrogen, phosphorus, and potassium (N—P—K) concentrations) in an effluent from the bioreactor can be determined using a nutrient analysis photometer such as, for example, a commercially available device sold under the name “Grow Master for Nutrient Analyses” (Model No. HI 83225) (Hanna® Instruments, Woonsocket, R.I.).

Use of other analytical methods/devices for determining the content of a nutrient in a composition such as an effluent from a bioreactor is within the ability of one of ordinary skill in the art.

Thus, in other aspects, the present invention provides a method for preparing a composition comprising nutrients, the method comprising:
determining a level of a nutrient in an effluent from a bio-reactor;
contacting the effluent with a source of the nutrient to adjust the level of the nutrient in the effluent; and
subjecting the effluent to conditions of time and temperature to inactivate substantially all microbes that may be present in the effluent.

In some embodiments, the steps of determining, contacting, and subjecting each may be performed one or more times as well as in any order relative to each other.

In one embodiment, the method comprises determining, contacting, and subjecting, wherein the step of determining is followed by the step of contacting, wherein the step of contacting is followed by the step of subjecting.

In another embodiment, the method comprises the steps of determining, contacting, and subjecting, wherein the step of subjecting is followed by the step of determining, wherein the step of determining is followed by the step of contacting.

In other embodiments, the method comprises the steps of determining, contacting, and subjecting, wherein the step of contacting is followed by the step of subjecting, wherein the step of subjecting is followed by the step of determining.

In another embodiment, the steps of determining and contacting are each performed after the step of subjecting.

In some embodiments, the steps of determining and contacting are each performed at least once, each before and after the step of subjecting.

In other embodiments, one may analyze and/or modify effluent conditions to optimize e.g., pH, electrical conductivity (EC), flow rate, other ingredients (e.g., adjuvants, other nutrients), and/or temperature.

For example, an effluent from a bioreactor may have a pH ranging from acidic to basic. Methods for adjusting the pH are well-known to those of skill in the art. Non-limiting examples of such adjustment methods include addition of base to increase pH or addition of acid to lower pH, as well as buffer systems. Acids and bases that can be used to adjust pH are familiar to one of skill in the art.

In one embodiment, the methods of the present invention further comprise adjusting the pH of an effluent.

In some embodiments, the pH and/or electrical conductivity (EC) of the effluent is analyzed and adjusted as appropriate e.g., to a pH range of about 4 to about 8, illustratively, to a pH1 of about 5 to about 7 and about 5.5 to about 6.5.

Examples of pH adjustment inputs include but are not limited to organic waste materials such as waste organic or inorganic acids (such as, but not limited to, citric acid, acetic acid, ascorbic acid, potassium phosphate, sodium phosphate, hydrochloric acid (HCl), phosphoric acid, nitric acid, sulfuric acid, etc.) and bases (such as, but not limited to, sodium hydroxide (NaOH), barium hydroxide (Ba₂OH), potassium hydroxide (KOH), etc.); they may also be derived from fruit and vegetable processing and nutrient extraction. Some of these materials can also serve as a chelating agent to increase the solubility of key macro and micronutrients in the aqueous solution.

Non-limiting examples of adjuvants that can be added to the effluent include but are not limited to surfactants, wetting agents, detergents, spreaders, stickers, retention aids, penetrators, synergists, activators, compatibility agents, humectants, drift retardants, bounces, shatter minimizers, and the like.

Testing/analyzing for nutrient content, pH, EC, and/or any other parameter may be performed one or more times as needed at any stage disclosed herein until the desired target formulation is achieved.

In other embodiments, the present invention provides a composition comprising nutrients that can be variably prepared and packaged (e.g., liquid bottling).

In some embodiments, a final container is utilized for packaging the composition comprising nutrients. In one embodiment, the final container is sealed (e.g., heat sealed, vacuum sealed, adhesively sealed).

In one embodiment, the final container is similar to conventional containers suitable for holding liquid fertilizers and that are typically sold commercially, in usual forms such as but not limited to shaped-containers based on, for example, polyethylene (PE) (e.g., high density polyethylene (HDPE)) containers, polyethylene terephthalate (PET) containers, metal containers, paper containers combined with metal foils or plastic films, or containers with cap assemblies that may be opened and closed.

In some embodiments, containers that cannot be retort-sterilized, may utilize sterilization processes wherein the containers are sterilized in advance at a high temperature for a period of time by use of e.g., a plate-type heat exchanger, and then cooled to a certain temperature, thereafter the containers being filled. Further, previously filled containers may be compounded and filled with another component under e.g., sterile conditions.

In other embodiments, the amount of the composition comprising nutrients may be provided in the final container in a concentrated liquid form. Concentration can be carried out by techniques known in the art including but not limited to controlled heating.

In one embodiment, the composition comprising nutrients may be dehydrated/lyophilized in accordance with known techniques and thus provided in the final container in powder form to which water or other liquid can be later added to form a liquid composition comprising the nutrients in either dilute or concentrated form.

In other embodiments, the present invention provides a method for preparing a composition comprising nutrients as disclosed herein, wherein the method further comprises one or more filtration steps.

The one or more filtration steps may be employed between any stage of processing described herein.

For example, one or more filtration steps may be performed before and/or after each step of determining, contacting, and subjecting as disclosed herein.

It is noted that a subsequent stage of filtration may be relatively more efficient than a preceding stage, since less amounts of particulates retained by a preceding filter remain in the filtrate solution. For example, a second stage filtration polishes the solution. Thus, for example the first stage filtration may remove more of the particulates/contamination that may be present in an upstream effluent.

The one or more filtration steps may be carried out using e.g., filters which, independently, have a pore or opening size ranging from about 1 to about 100 microns, illustratively, about 1 micron, about 5 microns, about 10 microns, about 15 microns, about 20 microns, about 25 microns, about 30 microns, about 35 microns, about 40 microns, about 45 microns, about 50 microns, about 55 microns, about 60 microns, about 65 microns, about 70 microns, about 75 microns, about 80 microns, about 85 microns, about 90 microns, about 95 microns, and about 100 microns.

In some embodiments, an effluent from a bioreactor is subjected to one or more filtration steps using e.g., filters which, independently, have a pore or opening size ranging from about 1 to about 100 microns, illustratively, about 1 micron, about 5 microns, about 10 microns, about 15 microns, about 20 microns, about 25 microns, about 30 microns, about 35 microns, about 40 microns, about 45 microns, about 50 microns, about 55 microns, about 60 microns, about 65 microns, about 70 microns, about 75 microns, about 80 microns, about 85 microns, about 90 microns, about 95 microns, and about 100 microns.

In another embodiment, the filter has a pore or opening size ranging from about 5 to about 25 microns.

In one embodiment, the one or more filtration steps comprise successive stages of filtration, using the same filter or different filters.

In some embodiments, a subsequent filtration step is used to further filter the filtrate of a preceding filtration step, wherein the filter used for the subsequent filtration step has a smaller pore or opening sizes than the filter of the preceding filtration step.

Filters for example, may be made of mesh, cloth, sintered glass, sintered metal, paper, plastic, polymer, or any material a person having skill in the art would find appropriate for their particular purpose. Such filters are commercially available, for example, from Whatman.

Exemplary methods of filtration including but not limited to leaf filtration, rotary drum filtration, rotary disk filtration, horizontal belt, horizontal table filtration and others are disclosed in, for example, Perry, R. H. and Green. D. W., Perry's Chemical Engineer's Handbook, 7th Edition, 1997 (McGraw-Hill, New York), which is herein incorporated by reference for its disclosure of filtration.

In some embodiments, the filter apparatus may be, for example, a vacuum filter apparatus, a rotating drum filter apparatus, a rotating vacuum drum filter apparatus, or other filter apparatus.

For example, in a filter apparatus using vacuum pressure, such as a vacuum filter apparatus or a rotating vacuum filter apparatus, vacuum pressure provided to the filter apparatus may be, for example, from between 5 mm Hg to about 30 mm Hg. In some embodiments, the vacuum pressure may be from 10 to about 20 inches Hg. Such vacuum filter apparatuses may be found commercially, for example, from Eimco-K. C. P. Ltd (Chennai, India) and Komline-Sanderson (Peapack, N.J.).

In filter apparatuses using rotation, such as a rotating drum filter apparatus, the filter apparatus may rotate, for example, at about 0.01 rpm or more, illustratively, from between about 0.01 rpm to about 30 or more rpms. In some embodiments, the filter apparatus is rotated at from between 0.05 to about 3 rpm.

In some embodiments, an effluent from a bioreactor is subjected to filtration prior to the step of subjecting.

For example, in one embodiment, a method of the present invention further comprises filtering an effluent obtained directly from the bioreactor using a first filter to form a first filtrate, and subjecting the first filtrate to conditions of time and temperature to inactivate substantially all microbes that may be present in the first filtrate thereby forming a sterile or substantially sterile first filtrate.

In one embodiment, the first filter is a 25 micron filter.

In other embodiments, the method further comprises filtering the sterile or substantially sterile first filtrate using a second filter to form a second filtrate.

In some embodiments, the second filter is a 25 micron filter.

In another embodiment, the second filtrate is subjected to filtration using a third filter to form a third filtrate.

In one embodiment, the third filter has a pore or opening size that is less than the pore or opening size of the second filter.

In other embodiments, the method further comprises determining a level of a nutrient in the third filtrate; and contacting the third filtrate with a source of the nutrient to adjust the level of the nutrient in the third filtrate to form a nutritionally supplemented composition.

In another embodiment, the nutritionally supplemented composition is subjected to filtration with a fourth filter to form a fourth filtrate.

In one embodiment, the fourth filter has a pore or opening size that is less than the pore or opening size of the second filter.

In other embodiments, the method further comprises bottling the fourth filtrate, wherein the fourth filtrate is the composition comprising nutrients.

In still further embodiments, the present invention provides a method for preparing a composition comprising nutrients, the method comprising:
holding an effluent from a bioreactor in a first container for at least a period of time sufficient to determine a level of a nutrient present in the effluent;
determining the level of the nutrient in the effluent;
contacting the effluent with a source of the nutrient to adjust the level of the nutrient in the effluent to form the composition comprising nutrients; and subjecting the composition to conditions of time and temperature to inactivate substantially all microbes that may be present in the composition.

In one embodiment, the subjecting step is performed in a second container.

In another embodiment, the method further comprises mixing the effluent and the source in the second container.

In some embodiments, the present invention provides a method for preparing a composition comprising nutrients, the method comprising:

subjecting an effluent from a bioreactor to conditions of time and temperature to inactivate substantially all microbes that may be present in the effluent;

determining the level of a nutrient in the effluent; and

contacting the effluent with a source of the nutrient to adjust the level of the nutrient in the effluent to form the composition comprising nutrients.

For example, in one embodiment, one or more holding tanks may be provided to receive an effluent from a bioreactor. In such an embodiment, the nutrient content of the effluent in a holding tank can be analyzed for the desired nutrient content such as, for example, nitrogen, phosphorus, and potassium (N—P—K) concentrations. For example, a sample of the effluent can be removed from the holding tank for chemical analysis to determine nutrient (e.g., N—P—K) content. Consequently, if any of the nutrient (e.g., N—P—K) values are low, they may be adjusted with their respective source equivalents to the desired level.

In other aspects, the present invention provides a composition comprising nutrients, wherein at least one nutrient present in the composition is derived from a food waste.

In one embodiment, the composition comprising nutrients is a plant food composition suitable for growing a plant.

In some embodiments, the total at least one nutrient derived from the food waste content of the composition is about 0.01% to about 100% by weight, illustratively, about 0.01% to about 100%, about 0.1% to about 95%, about 1% to about 90%, about 5% to about 85%, about 10% to about 80%, about 15% to about 75%, about 20% to about 70%, about 25% to about 65%, about 30% to about 60%, about 35% to about 55%, and about 40% to about 50% by weight.

In another embodiment, the total at least one nutrient derived from the food waste content of the composition is at least about 0.01% by weight, illustratively, at least about 0.1%, at least about 1%, at least about 2.5%, at least about 5%, at least about 7.5%, at least about 10%, at least about 12.5%, at least about 15%, at least about 17.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, at least about 60%, at least about 62.5%, at least about 65%, at least about 67.5%, at least about 70%, at least about 72.5%, at least about 75%, about 77.5%, at least about 80%, at least about 82.5%, at least about 85%, at least about 87.5%, at least about 90%, at least about 92.5%, at least about 95%, at least about 97.5%, and about 100% by weight.

In other embodiments, the composition comprising nutrients is a plant food composition suitable for use for growing a plant, wherein the composition is prepared according to the methods and systems disclosed herein.

In one embodiment, the present invention provides a composition comprising nutrients, wherein the composition is prepared by a method comprising:

subjecting an effluent from a bio-reactor to conditions of time and temperature to inactivate substantially all microbes that may be present in the effluent; and

optionally, contacting the effluent with a source of a nutrient to adjust a level of the nutrient in the effluent.

Generally, a plant food composition of the present invention may be used in a variety of systems for growing plants, including but not limited to a hydroponic system as well as conventional soil-based systems.

The term “hydroponic” as used herein refers to a method/system for growing plant material in solution rather than in conventional soil. A variety of hydroponic systems are available and the present invention is not limited for any particular type of system. Thus, the term “hydroponic” unless specifically stated otherwise includes true hydroponics, semi- or quasi-hydroponic environments such as in admixtures with some amounts of soil, aeroponics, aquaculture, aquaponic, drip irrigation, food and drain, wick, Nutrient Film Technique (NFT) systems and all other hydroponic growth systems.

In one embodiment, the composition comprising nutrients is an aqueous plant food composition (e.g., a liquid fertilizer) suitable for use in a hydroponic system.

In some embodiments, the nutrient elements are supplied to a plant as an aqueous nutrient solution, for example through an irrigation system.

In other embodiments, the nutrient solution contains at least the nutrient elements nitrogen, phosphorus, and/or potassium under ion form.

A plant food composition of the present invention can be tailored for use for application to any plant or part thereof, including for plant roots and their cuttings, and is in particular useful for produce, in particular vegetables or fruits, or for flowers.

For example, the plant food composition can inter alia be suitable for growing leafy vegetables, including leafy greens such as, for example, lettuce and spinach, for tubers, like potato or sweet potato, for roots, such as celeriac, for shoots, such as witloof, or for mushrooms.

The plant food composition can furthermore be used for growing fruits, such as, for example, apple, banana, avocado, peach, pear, apricot, mango, eggplant and for flowers or flower stems, such as, for example, gerbera stems, chrysanthemum flowers, artichoke bottoms, etc.

In one embodiment, the composition comprising nutrients is an aqueous composition, wherein the aqueous composition comprises at least about 0.01% of one or more of nitrogen (N), phosphorus (P), and potassium (K) by weight, illustratively, at least about 0.01%, at least about 0.1%, at least about 0.5%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.1%, at least about 3.2%, at least about 3.3%, at least about 3.4%, at least about 3.5%, at least about 3.6%, at least about 3.7%, at least about 3.8%, at least about 3.9%, at least about 4%, about 4.1%, at least about 4.2%, at least about 4.3%, at least about 4.4%, at least about 4.5%, at least about 4.6%, at least about 4.7%, at least about 4.8%, at least about 4.9%, at least about 5%, about 5.1%, at least about 5.2%, at least about 5.3%, at least about 5.4%, at least about 5.5%, at least about 5.6%, at least about 5.7%, at least about 5.8%, at least about 5.9%, at least about 6%, about 6.1%, at least about 6.2%, at least about 6.3%, at least about 6.4%, at least about 6.5%, at least about 6.6%, at least about 6.7%, at least about 6.8%, at least about 6.9%, at least about 7%, about 7.1%, at least about 7.2%, at least about 7.3%, at least about 7.4%, at least about 7.5%, at least about 7.6%, at least about 7.7%, at least about 7.8%, at least about 7.9%, and at least about 8% by weight of one or more of nitrogen (N), phosphorus (P), and potassium (K).

In some embodiments, the composition comprising nutrients is an aqueous composition, wherein the aqueous composition comprises a total nitrogen (N) content of at least about 5% by weight.

In other embodiments, the composition comprising nutrients is an aqueous composition, wherein the liquid composition comprises a total phosphorus (P) content of at least about 5% by weight.

In one embodiment, the composition comprising nutrients is an aqueous composition, wherein the aqueous composition comprises a total potassium (K) content of at least about 5% by weight.

In another embodiment, the composition comprising nutrients is an aqueous composition comprising a total combined nitrogen, phosphorus, and potassium (N—P—K) content of at least about 5% by weight.

In some embodiments, the composition comprising nutrients is an aqueous composition, wherein the aqueous composition comprises a total nitrogen (N) content of at least about 6% by weight.

In other embodiments, the composition comprising nutrients is an aqueous composition, wherein the aqueous composition comprises a total phosphorus (P) content of at least about 6% by weight.

In one embodiment, the composition comprising nutrients is an aqueous composition, wherein the aqueous composition comprises a total potassium (K) content of at least about 6% by weight.

In another embodiment, the composition comprising nutrients is an aqueous composition comprising a total combined nitrogen, phosphorus, and potassium (N—P—K) content of at least about 6% by weight.

In some embodiments, the composition comprising nutrients is an aqueous composition, wherein the aqueous composition comprises a total nitrogen (N) content of about 1 mg/L to about 100,000 mg/L, illustratively, about 1 mg/L to about 100,000 mg/L, about 10 mg/L to about 90,000 mg/L, about 50 mg/L to about 90,000 mg/L, about 100 mg/L to about 80,000 mg/L, about 500 mg/L to about 70,000 mg/L, about 1000 mg/L to about 60,000 mg/L, about 5000 mg/L to about 50,000 mg/L, about 10,000 mg/L to about 40,000 mg/L, and about 20,000 mg/L to about 30,000 mg/L.

In other embodiments, the composition comprising nutrients is an aqueous composition, wherein the aqueous composition comprises a total nitrogen (N) content of at least about 1 mg/L, illustratively, at least about 1 mg/L, at least about 10 mg/L, at least about 100 mg/L, at least about 500 mg/L, at least about 1000 mg/L, at least about 5000 mg/L, at least about 10,000 mg/L, at least about 20,000 mg/L, at least about 30,000 mg/L, at least about 40,000 mg/L, at least about 50,000 mg/L, at least about 60,000 mg/L, at least about 70,000 mg/L, at least about 80,000 mg/L, at least about 90,000 mg/L, and at least about 100,000 mg/L.

In one embodiment, the composition comprising nutrients is an aqueous composition, wherein the aqueous composition comprises a total phosphorus (P) content of about 0.1 mg/L to about 100,000) mg/L, illustratively, about 0.1 mg/l, to about 10,000 mg/L, about 1 mg/L to about 9,000 mg/L, about 50 mg/L to about 8,000 mg/L, about 100 mg/L to about 7,000 mg/L, about 500 mg/L to about 5,000 mg/L, about 1000 mg/L to about 4,000 mg/L, and about 200 mg/L to about 3,000 mg/L.

In another embodiment, the composition comprising nutrients is an aqueous composition, wherein the aqueous composition comprises a total phosphorus (P) content of at least about 0.1 mg/L, illustratively, at least about 0.1 mg/L, at least about 1 mg/L, at least about 10 mg/L, at least about 100 mg/L, at least about 500 mg/L, at least about 1000 mg/L, at least about 2000 mg/L, at least about 3,000 mg/L, at least about 4,000 mg/L, at least about 5,000 mg/L, at least about 6,000 mg/L, at least about 7,000 mg/L, at least about 8,000 mg/L, at least about 9,000 mg/L, at least about 10,000 mg/L, at least about 20,000 mg/L, and at least about 30,000 mg/L.

In some embodiments, the composition comprising nutrients is an aqueous composition, wherein the aqueous composition comprises a total potassium (K) content of about 1 mg/L to about 100,000 mg/L, illustratively, about 1 mg/L to about 100,000 mg/L, about 10 mg/L to about 90,000 mg/L, about 50 mg/L to about 90,000 mg/L, about 100 mg/L to about 80,000 mg/L, about 500 mg/L to about 70,000 mg/L, about 1000 mg/L to about 60,000 mg/L, about 5000 mg/L to about 50,000 mg/L, about 10,000 mg/L to about 40,000 mg/L, and about 20,000 mg/L to about 30,000 mg/L.

In other embodiments, the composition comprising nutrients is an aqueous composition, wherein the aqueous composition comprises a total potassium (K) content of at least about 1 mg/L, illustratively, at least about 1 mg/L, at least about 10 mg/L, at least about 100 mg/L, at least about 500 mg/L, at least about 1000 mg/L, at least about 5000 mg/L, at least about 10,000 mg/L, at least about 20,000 mg/L, at least about 30,000 mg/L, at least about 40,000 mg/L, at least about 50,000 mg/L, at least about 60,000 mg/L, at least about 70,000 mg/L, at least about 80,000 mg/L, at least about 90,000 mg/L, and at least about 100,000 mg/L.

In some embodiments, the composition comprising nutrients comprises at least about 0.1% by weight of its total nutrient content derived from a food waste, illustratively, at least about: 0.1, 0.2, 0.4, 0.6, 0.8, 1, 5, 10, 25, 30, 35, 40, 45, 50, 55, 60, 65, 70, 75, 80, 85, 90, or 95% by weight, or about 100% by weight.

In other embodiments, the composition comprising nutrients is a liquid fertilizer.

In other aspects, the present invention provides a container comprising a composition of the present invention.

In one embodiment, the present invention provides a container comprising an effluent as disclosed herein.

In some embodiments, the container is a storage container suitable for holding liquids for a period of time.

Suitable storage containers include, but are not limited to, tanks, collapsible tanks, drums (e.g., metal, plastic), liquid bulk containers, closed-head and open-head containers, pillow storage tanks, bladder tanks and carboys.

In one embodiment, the container is a stainless steel tank.

In another embodiment, the container is a final container (e.g., bottle) as disclosed herein.

In other aspects, the present invention provides an integrated product comprising a container comprising a composition comprising nutrients and instructions for use, wherein the container and the composition are as disclosed herein.

In other aspects, the present invention provides a system for the production of a composition comprising nutrients.

In some embodiments, the system comprises a first container for receiving an effluent from a bio-reactor.

In other embodiments, the system further comprises the bio-reactor and a second container for receiving a feed solution from the first container, wherein the first container is operatively connected to the bio-reactor for receiving the effluent existing the bio-reactor, and wherein the second container is operatively connected to the first container for receiving the feed solution from the first container.

In another embodiment, the first container is configured for receiving one or more additives from a source, wherein the additives comprise a nutrient, and wherein the second container is configured for subjecting the effluent to conditions of time and temperature to inactivate substantially all microbes that may be present in the effluent.

In other embodiments, the first container is configured for subjecting the effluent to conditions of time and temperature to inactivate substantially all microbes that may be present in the effluent, wherein the second container is configured for receiving one or more additives from a source, wherein the additives comprise a nutrient.

A system according to the present invention may include one or more additional stations/components integrated thereto including but not limited to filtration, bottling (e.g., using an apparatus/instrument for bottling), final sterilization, etc., whereby food waste is utilized to prepare compositions comprising nutrients, in particular liquid compositions comprising nutrients for growing a plant.

A variety of bottling/packaging methods/devices (e.g., using any apparatus/instrument used for bottling) are known in the art including but not limited to automated, semi-automated, and manual methods/apparatuses/devices.

In one embodiment, packaging of the final liquid product comprises pumping the final product from a bulk container to a final product container.

For example, in some embodiments, the final product to be packaged is hand pumped from an Intermediate Bulk Container (IBC) into the final product container.

In other embodiments, the systems disclosed herein accordingly provide for a multistep system that is assembled and/or integrated at a single site or location that includes the bioreactor as well as other downstream components for processing of the effluent to form the final composition comprising nutrients.

In another embodiment, the present invention provides a system for preparing a composition comprising nutrients, the system comprising:

a bioreactor;

a first container for holding an effluent from the bioreactor;

a mixing container for mixing an aqueous solution placed therein; and

a bottling station.

In one embodiment, the bioreactor, the first container, the mixing container, and the bottling station (e.g., an apparatus/instrument used for bottling) are operatively connected, wherein the first container is adapted to receive an effluent from the bioreactor.

In another embodiment, the system further comprises a reverse osmosis unit, wherein the reverse osmosis unit is in fluid communication with the bioreactor is configured to filter the aqueous solution entering the bioreactor.

In some embodiments, the system further comprises one or more filtration units between the bioreactor and the first container, the first container and the mixing container, and the missing container and the bottling station.

In other embodiments, the present invention provides for a system comprising modular components where one or more of which may be transported and/or processed at one or more different locations.

For example, in some embodiments, the effluent that forms in the bioreactor is placed in a first container that is capable of being sealed and transported to a different location for further processing e.g., for filtering, determining, contacting, mixing, and/or subjecting of the effluent as disclosed herein.

In other embodiments, in addition to use on transport vehicles, which may include a variety of modes such as, for example, truck, rail, ship or air, the first container may also be suitable for standalone storage for a period of time before further processing of its content is performed.

Various modifications of the present invention, in addition to those described herein, will be apparent to those skilled in the art from the foregoing description. Such modifications are also intended to fall within the scope of the appended claims.

The present invention will be illustrated in more detail by way of Examples, but it is to be noted that the invention is not limited to the Examples.

### EXAMPLES

### Example 1

### Process Outline

In accordance with one embodiment of the present invention, nutrient rich effluent is created by food waste liquefier in batch form. From there, fiberglass liquid storage tanks will be used for temporary storage of the effluent before proceeding to sterilizing/stabilizing the effluent with addition of a composition comprising a biocide/preservative (e.g., HydroStat™, sulfuric acid, phosphoric acid, and/or calcium additive(s), etc.) to stabilize and sterilize the effluent, inactivating pathogens and ensuring that all “live” microbes are stabilized. The sterilized liquid effluent is added to a larger industrial mixing vessel where additives, also referred to as nutrient sources, are added and mixed to complete the product's formulation. Mixing is performed until the targeted product formulation is reached. The final liquid product is then added to fiberglass liquid storage tanks where the finished product will be stored. The final liquid product is bottled using any apparatus/instrument used for bottling.

### Example 2

### Process Outline

In accordance with another embodiment of the present invention, nutrient rich effluent is created by food waste liquefier in batch form. From there, fiberglass liquid storage tanks will be used for temporary storage of the effluent before proceeding to sterilizing/stabilizing the effluent in an industrial autoclave tank. An industrial heated autoclave tank with a heat source is used to stabilize and sterilize the effluent, inactivating pathogens and ensuring that all “live” microbes are stabilized. The sterilized liquid effluent is added to a larger industrial mixing vessel where additives, also referred to as nutrient sources, are added and mixed to complete the product's formulation. Mixing is performed until the targeted product formulation is reached. The final liquid product is then added to fiberglass liquid storage tanks where the finished product will be stored. The final liquid product is bottled using any apparatus/instrument used for bottling.

### Example 3

### Process for Preparing a Nutrient Rich Aqueous Composition

In accordance with some embodiments of the present invention, the starting materials for the process include a mixture of organic input sources, including anaerobic microbial flora and fauna, pH adjustments inputs, and nutrient sources.

The organic waste is gathered and sorted to remove unwanted waste, and to tailor the nutrient content before it is put in the food waste liquefier tank. Nutrient source inputs will vary in nutrient content according to the origin of the organic waste material. Thus, if the nutrient content is less than optimal, the effluent will then be adjusted by the selective addition of organic, naturally occurring sources of nitrogen (N), phosphorus (K), and potassium (K).

The organic waste is fed into a food waste liquefier. Once sealed, water spray and agitation work synergistically with a blend of microbial flora, fauna, humic acid, and woodchips to reduce the waste into a nutrient rich effluent. Next, the effluent is moved to holding tanks where its nutrient content will be analyzed for the desired nitrogen, phosphorus, and potassium (N—P—K) concentrations.

Consequently, if any of the N—P—K values are low, they will be adjusted with their respected naturally occurring source equivalents. In addition, the pH of the effluent will be tested and controlled if needed to a pH range of 5-7. Examples of pH adjustment inputs are organic waste materials such as waste organic acids (citric, acetic, etc). Or, they can be derived from fruit and vegetable processing and nutrient extraction. These materials can also serve as a chelating agent to increase the solubility of key macro and micro nutrients in the effluent.

Afterwards, the effluent will then be retested for its N—P—K concentration and will proceed to bottling, once the desired quality has been achieved.

If the nutrient rich effluent is sufficient per a targeted or desired N—P—K value, the effluent will proceed to additional processing by sterilizing, stabilizing, mixing, and bottling until it is ready for distribution to the end consumer.

What is claimed is:

1. A method for preparing a composition comprising nutrients, the method comprising:
subjecting an effluent from a bio-reactor to conditions of time and temperature to inactivate substantially all microbes that may be present in the effluent; and
optionally, contacting the effluent with a source of a nutrient to adjust a level of the nutrient in the effluent.

2. The method of claim 1, wherein the step of contacting is performed prior to the subjecting step.

3. A method for preparing a composition comprising nutrients, the method comprising:
determining a level of a nutrient in an effluent from a bio-reactor;
contacting the effluent with a source of the nutrient to adjust the level of the nutrient in the effluent; and
subjecting the effluent to conditions of time and temperature to inactivate substantially all microbes that may be present in the effluent.

4. The method of claim 3 further comprising:
adjusting the pH of the effluent.

5. The method of claim 3 further comprising:
holding the effluent in a first container for at least a period of time sufficient to determine the level of the nutrient present in the effluent.

6. The method of claim 5, wherein the contacting step occurs in the first container, and wherein the subjecting step occurs in a second container.

7. The method of claim 6 further comprising:
mixing the effluent and the source in the second container.

8. The method of claim 1, wherein the bioreactor is a food waste liquefier, and wherein the nutrient is a plant nutrient.

9. The method of claim 1, wherein the nutrient is selected from the group consisting of: H₂PO₄⁻, SO₄²⁻, NH₄⁺, Ca^{2+,} K⁺ and Mg²⁺.

10. The method of claim 1, wherein the effluent is decomposed food waste, and wherein the bio-reactor is a food waste liquefier.

11. The method of claim 1, wherein the bio-reactor is a food-waste liquefier, and wherein the effluent is an aqueous effluent from the food-waste liquefier.

12. The method of claim 1, wherein the composition is an aqueous solution.

13. A composition comprising nutrients prepared according to the method of claim 1.

14. The composition of claim 13, wherein the composition is suitable for use in a system for growing a plant.

15. The composition of claim 14, wherein the system for growing the plant comprises a hydroponic system.

16. The composition of claim 14, wherein the system for growing the plant comprises a soil-based system.

17. A composition comprising a food waste extract and a plant-based ingredient, wherein the composition is suitable for use in a system for growing a plant.

18. The composition of claim 13, wherein the composition is an aqueous solution.

19. The composition of claim 18, wherein the composition comprises a pH of about 5 to about 7.

20. A container comprising the composition of claim 11.

21. The container of claim 20, wherein the container is a bottle.

22. A system comprising:
a first container for receiving an effluent from a bio-reactor that composts a bio-compostable material.

23. The system of claim 22 further comprising the bio-reactor.

24. The system of claim 22 further comprising a second container for receiving a feed solution from the first container.

25. The system of claim 24, wherein the first container is operatively connected to the bio-reactor for receiving the effluent existing the bio-reactor, and wherein the second container is operatively connected to the first container for receiving the feed solution from the first container.

26. The system of claim 24, wherein the first container is configured for receiving one or more additives from a source, and wherein the second container is configured for subjecting the effluent to conditions of time and temperature to inactivate substantially all microbes that may be present in the effluent.
